(19) United States
(12) Patent Application Publication
(10) Pub. No.: US 2025/0134800 A1
(43) Pub. Date: May 1, 2025

(54) Biomass Extract For Skin Applications

(71) Applicant: EMBION TECHNOLOGIES SA, Etoy (CH)

(72) Inventors: Sviatlana SIANKEVICH, Fechy (CH); Georgios SAVOGLIDIS, Fechy (CH)

(21) Appl. No.: 18/835,395

(22) PCT Filed: Feb. 2, 2023
(86) PCT No.: PCT/EP2023/052605

§ 371 Date: Aug. 2, 2024

(30) Foreign Application Priority Data
Feb. 3, 2022 (EP) 22155093.2

(51) Int. Cl. A61K 8/9794; A61K 8/9728; A61Q 17/00; A61Q 19/00; A61Q 19/08; C08F 236/22

(52) U.S. Cl. CPC A61K 8/9794; A61K 8/9728; A61Q 17/005; A61Q 19/007; A61Q 19/08; C08F 236/22

(57) ABSTRACT

The invention relates to compositions having prebiotic and antipathogenic properties and use thereof for skin cosmetic purposes, such as regeneration and protection of the skin.

### FIELD OF THE INVENTION

The invention relates to compositions having prebiotic and antimicrobial properties and use thereof for skin cosmetic purposes, such as regeneration and protection of the skin.

### BACKGROUND OF THE INVENTION

Skin in mammals is considered as one of the largest organs in the body, especially one which has the largest surface area. Skin forms the first line of defence against microorganisms which may invade the body though the air, water, food or material that come in contact with the body. When the body is infected on the skin or systemically, traditional approach to such hygiene problems has been to treat the skin/body with antimicrobial actives that reduce or kill the germs. Recent research indicates that a lot of the bacteria that permanently reside on the skin (called skin commensal bacteria) do not actually cause infections, rather they are beneficial bacteria that protect the skin against disease causing pathogens. Thus, there is a trend in moving away from the approach of treating the skin with broad spectrum antimicrobial actives to kill all microoganisms present on skin (or any other part of the body) as a means of treating infections. Rather, the approach is more towards targeted or selective inhibition/killing of the desired microorganism to the exclusion of the skin commensal organism and/or booting the number and functionality of skin commensal bacteria through use of prebiotics.

The health and cosmetic benefits of providing a healthy, balanced skin microbiome are only recently becoming better understood. As a result, only a limited number of suitable prebiotic agents have been identified for use on skin. In addition, conventional prebiotic agents are typically administered orally, for example, as part of a nutritional supplement regimen. While oral ingestion may be suitable for delivering prebiotic agents to the GI tract, it may not be the best way to deliver a prebiotic to the microbiota found on the skin.

Accordingly, there is a need to improve the health and/or appearance of human skin by providing a composition that exhibits prebiotic activity on one or more skin commensal microorganisms. At the same time there is a need to control undesirable microbial activity.

### SUMMARY OF THE INVENTION

An aspect of the present invention provides a method for producing a prebiotic composition, the method comprising the steps of:
a) providing biomass;
b) optionally determining lipids, proteins and/or carbohydrates contents in the biomass;
c) optionally pre-treating the biomass, wherein the optional pre-treatment is hot water pre-treatment at about 90° C.;
d) optionally removing lipids and/or proteins from the biomass;
e) contacting the biomass with a catalyst in the presence of water and/or an organic solvent to form a reaction mixture, wherein the catalyst is an ionic polymer or a combination of ionic polymers, the ionic polymer network, a solid-supported ionic polymers and/or a polymer membrane incorporating ionic polymers;
f) heating the reaction mixture between 100° C. to 150° C. and degrading the biomass in the reaction mixture to produce a liquid phase and a solid phase, wherein the liquid phase includes the prebiotic composition, and the solid phase includes residual biomass;
g) cooling the reaction mixture to room temperature;
h) isolating at least a portion of the liquid phase from the solid phase;
i) optionally adding one or more compounds selected from bentonite, charcoal, zeolite, amorphous silica and/or ion exchange solvent to the isolated liquid phase; and
j) recovering the prebiotic composition from the isolated liquid phase.
   wherein the ionic polymer (IP) consists of a monomer of formula I

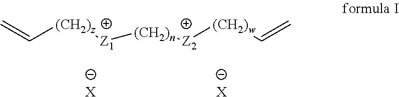

or consists of a first monomer of formula I

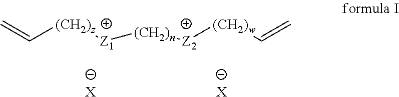

and at least one second monomer selected from the group consisting of

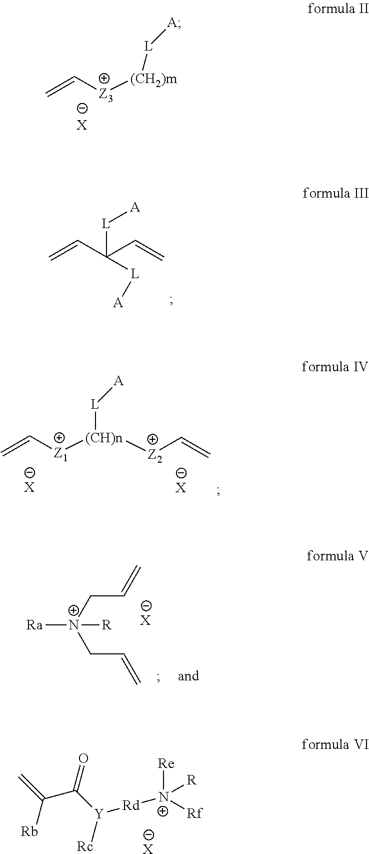

wherein
n and m are independently selected from 1, 2, 3, 4, 5, 6;
z and w are independently selected from 0, 1, 2, 3;
Z₁, Z₂ and Z₃ are cations each independently selected from the group comprising:

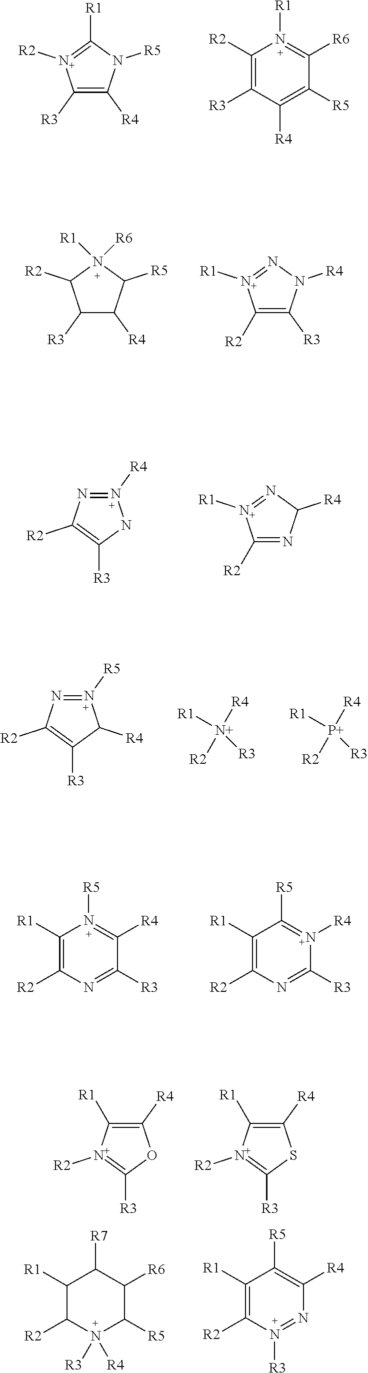

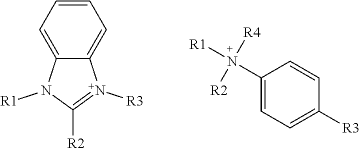

R1, R2, R3, R4, R5, R6 and R7 are each independently selected from the group comprising a bond, H, C₁-C₆ alkyl, C₁-C₆ allyl, —CH₂—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group comprising H, —SO₃H, —COOH, —[P(═O)(OH)₂], —O—SO₃H, —O—COOH, —O—[P(═O)(OH)₂];
A is an optional acidic group and each occurrence of A, if present, is independently selected from the group comprising H, —SO₃H, —COOH, —[P(═O)(OH)₂], —O—SO₃H, —O—COOH, —O—[P(═O)(OH)₂], —CH₂—COOH, provided that when z and w are 0, A is present in formula IV;
X⁻ is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate;
Ra is C₁-C₂₄ alkyl;
Rb and Rc are each independently selected from the group comprising H and CH₃ or absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is selected from the group comprising C₁-C₂₄ alkyl and C₅-C₁₀ aryl or is absent.
wherein the ionic polymer network comprises cross-linked the one or more ionic polymers (IP);
wherein the solid support has at least one surface comprising the one or more ionic polymers (IP) or the ionic polymer network;
wherein the polymer membrane incorporates the one or more ionic polymers (IP) or the ionic polymer network.

Another aspect of the present invention provides a prebiotic composition obtained by the method of the invention.

Another aspect of the present invention provides a topical cosmetic composition comprising:
an effective amount of the prebiotic composition of the invention, and
at least one dermatologically acceptable carrier.

Another aspect of the present invention provides a use of the prebiotic composition of the invention or the topical composition of the invention for non-therapeutic treatment of the skin.

Another aspect of the present invention provides a use of the prebiotic composition of the invention or the topical composition of the invention for modulating the growth of at least one commensal bacterium of the skin, the method comprising applying an effective amount of the prebiotic composition of the invention or the topical composition of the invention to the skin.

Another aspect of the present invention provides a use of the prebiotic composition of the invention or the topical composition of the invention for preventing dysbiosis on an external surface of a human or animal body.

Another aspect of the present invention provides a use of the prebiotic composition of the invention or the topical composition of the invention for stimulating immune skin system.

### BRIEF DESCRIPTION OF THE FIGURES

FIG. 1 shows gene expression by qRT-PCR: inhibitory study vs S. aureus.

FIG. 2 shows gene expression for the prebiotic study vs S. epidermidis after 2 days.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

“Cosmetic composition” means a composition suitable for topical application on mammalian, preferably human, skin and/or other keratinous tissue such as hair and nails, which is intended to improve the condition and/or appearance of the skin or keratinous tissue or otherwise provide a skin care benefit. Topical means the surface of the skin or other keratinous tissue. Cosmetic composition includes any colour cosmetic, nail, or skin care product. “Skin care” means regulating and/or improving skin condition. Some nonlimiting examples of skin care benefits and improvement of the condition and/or appearance of the skin include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin texture or smoothness, improving skin exfoliation or desquamation, plumping the skin, improving skin barrier properties, improve skin tone, reducing the appearance of redness or skin blotches, and/or improving the brightness, radiancy, or translucency of skin. Some non-limiting examples of cosmetic compositions include products that leave colour on the face, such as foundation, mascara, concealers, eye liners, brow colours, eye shadows, blushers, lip sticks, lip balms, face powders, solid emulsion compact, and the like. “Skin care products” include, but are not limited to, skin creams, moisturizers, lotions, and body washes.

“Dermatologically acceptable carrier” means a carrier that may be applied topically to skin or keratinous tissue. The dermatologically acceptable carrier may be in a wide variety of forms such as, for example, simple solutions (water-based or oil-based), solid forms (gels or sticks) and emulsions (water-in-oil or oil-in-water).

“Effective amount” means a sufficient amount of the specified component/composition to have the specified properties under the specified conditions. For example, an effective amount of a prebiotic composition means an amount sufficient to cause a desired increase in the metabolite level and/or bacterial counts of one or more selected microorganisms in vitro and/or in vivo.

“Prebiotic” means any substance or combination of substances that can be utilized as a nutrient by a selected microorganism (e.g., a skin commensal microorganism), can induce the growth and/or activity of a selected microorganism, can induce the replication of a selected microorganism, can be utilized as an energy source by a selected microorganism, and/or can be utilized by a selected microorganism for the production of biomolecules (i.e. RNA, DNA, and proteins). Non-limiting examples of prebiotics include mucopolysaccharides, oligosaccharides such as galactooligosaccharides (“GOS”), polysaccharides, amino acids, vitamins, nutrient precursors, harvested metabolic products of biological organisms, lipids, and proteins.

“Replication” means the division of a microorganism into daughter cells (e.g. by mitosis or binary fission).

“Skin” means one or more of the epidermis, dermis, and hypodermis (i.e., subcutis), hair follicles, hair roots, hair bulbs, the ventral epithelial layer of the nail bed (lectulus), sebaceous glands and perspiratory glands (eccrine and apocrine). Skin is mammalian skin, preferably human skin. In some embodiment, the skin is an external surface of a human or animal body.

“Skin commensal microorganisms” or “skin commensal bacteria” or “commensal bacteria” means prokaryotes and eukaryotes that may colonize (i.e., live and multiply on human skin) or temporarily inhabit human skin in vitro and/or in vivo. Commensal bacteria act on the host's immune system to induce protective responses that prevent colonization and invasion by pathogens.

“Topical” and variations thereof refer to compositions that are intended to be applied directly to the outer surface of the skin or other keratinous tissue.

Unless otherwise noted, the term “microbiome” refers to the collective genomes of the microbes (composed of bacteria, bacteriophage, fungi, protozoa and viruses) that live inside and on the human body.

An “allyl” group is a substituent with the structural formula H₂C═CH—CH₂R, where R is the rest of the molecule.

The term “monomer” refers to a molecule that can undergo polymerization or copolymerization thereby contributing constitutional units to the essential structure of a macromolecule (a polymer).

“Cross-linking”, as used herein, refers to the attachment of two or more monomers, oligomers or longer polymer chains by bridges of a cross-linker, such as an element, molecular group, a compound, or another oligomer or polymer. Cross-linking can result in a polymeric network (which can be two-dimensional or three-dimensional) where the polymer subunits are interconnected with multiple cross-linking agents and without free ends. Cross-linking may take place upon exposure to a stimulus, such as heat or light. As a result, some cross-linking processes occur at increased temperature, and some may also occur at room temperature or at lower temperature. As cross-linking density is increased, the properties of a material can be changed from thermoplastic to thermosetting.

As used herein, “phenolic compounds” are a group of small molecules characterized by their structures having at least one phenol unit.

An aspect of the present invention provides a method for biomass hydrolysis into a prebiotic composition with excellent antimicrobial and prebiotic properties.

In an embodiment, the present invention provides a method for producing a prebiotic composition from biomass, the method comprising the steps of:
a) providing biomass;
b) optionally determining lipids, proteins and/or carbohydrates contents in the biomass;
c) optionally pre-treating the biomass, wherein the optional pre-treatment is hot water pre-treatment at about 90° C.;
d) optionally removing lipids and/or proteins from the biomass;
e) contacting the biomass with a catalyst in the presence of water and/or an organic solvent to form a reaction mixture, wherein the catalyst is an ionic polymer or a combination of ionic polymers, the ionic polymer network, a solid-supported ionic polymers and/or a polymer membrane incorporating ionic polymers;
f) heating the reaction mixture between 100° C. to 150° C. and degrading the biomass in the reaction mixture to produce a liquid phase and a solid phase, wherein the liquid phase includes the prebiotic composition, and the solid phase includes residual biomass;
g) cooling the reaction mixture to room temperature;
h) isolating at least a portion of the liquid phase from the solid phase;
i) optionally adding one or more compounds selected from bentonite, charcoal, zeolite, amorphous silica and/or ion exchange solvent to the isolated liquid phase; and
j) recovering the prebiotic composition from the isolated liquid phase.

In one embodiment, the step e) contacting the biomass with a catalyst to form a reaction mixture consists in adding water and/or an appropriate organic solvent and an effective amount of the catalyst to the biomass to form a reaction mixture, wherein the catalyst is an ionic polymer of the invention or a combination of ionic polymers of the invention, the ionic polymer network of the invention, a membrane incorporating ionic polymers of the invention and/or a solid-supported ionic polymers of the invention; and degrading step f) consists in heating the reaction mixture of step e) during appropriate time and subsequently cooling to room temperature (typically 20-25° C.).

In some embodiments of the method for producing the prebiotic composition from biomass, the method further comprises applying a pressure of N₂ or CO₂ during the degrading step f). The pressure may range from 20 bar to 300 bar, preferably from 20 to 150 bar.

In some embodiments of the method for producing the prebiotic composition from biomass, the step i) is performed, i.e. it is not optional.

The term “biomass,” as used herein, refers to living or dead biological material that can be used in the method for producing the prebiotic composition of the invention. In some embodiments, the biomass is selected from the group comprising spent yeast slurry, spent barley, brewer's spent grains, pellets, nuts or (cattle) cake; a crop or crop residue: corn, soybeans, sorghum, oats, barley, copra, chaff, husks or hulls, sugar beet waste; products of decortication; fish meal; meat and bone meal; molasses; oil cake and press cake; oligosaccharides; conserved forage plants: silage; seaweed; seeds and grains, either whole or prepared by crushing, milling etc.; sprouted grains and legumes; yeast extract. Preferably, the biomass is yeast slurry or brewers' spent grains. Most preferably the biomass is brewer's spent grains. The term “brewer's spent grain (BSG)” is a food waste that is a by-product of the brewing industry and is obtained as a mostly solid residue after wort production in the brewing process. The term “yeast slurry” is typically used in brewing industry to describe the pasty mixture of yeast and a liquid.

Optionally, prior to any use, carbohydrates and/or lipids contents are determined in the biomass based on the standard methods. Lipids can be determined/extracted using Folch method (Folch J, Lees M, Stanley, G H S, 1957, 226, 497-509) involving a mixture of methanol, chloroform and water (2:1:0.8, v/v/v), and phase separation afterwards. Determination of carbohydrates is performed according, for example, NREL protocol for “Determination of Structural Carbohydrates and Lignin in Biomass”. For example, 1 ml of 72% sulfuric acid was added to 100 mg of biomass. The slurry was stirred for 1 h at 30° C., followed by addition of 28 ml of deionized water. Mixture was autoclaved at 120 C for 1 h, cooled to room temperature and was used for sugar analysis by HPLC and acid-soluble lignin determination using UV-spectrophotometry at 205 nm wavelength. The same hydrolysate was used for proteins analysis according to the Bradford protein assay. The residue from acid hydrolysis was washed with 100 mL of water and then dried at 105° C. to determine Klason lignin.

The optional pre-treatment of the biomass, used in the methods described herein, uses one or more methods selected from the group consisting of washing, solvent-extraction, solvent-swelling, comminution, milling, steam pre-treatment, explosive steam pre-treatment, dilute acid pre-treatment, hot water pre-treatment (at about 90° C.), alkaline pre-treatment, lime pre-treatment, wet oxidation, wet explosion, ammonia fibre explosion, organosolvent pre-treatment, biological pre-treatment, ammonia percolation, ultrasound, electroporation, microwave, supercritical CO₂, supercritical H₂O, ozone, and gamma irradiation. The optional pre-treatment of the biomass includes for example the milling of the biomass. To overcome the obstacle of the reaction rate being limited by the surface reaction and mass transfer, a pre-treatment processes of the biomass via ball milling, which leads to a reduction in crystallinity and an increase in the specific surface area of cellulosic material, is highly recommended. Depending on performed mechanical ball milling of biomass there is a decrease in structural particle size, reduction of the degree of polymerization of cellulose, and an increase in the amorphous content of cellulose.

In an embodiment of the method for producing a prebiotic composition of the invention, the optional pre-treating of the biomass (step c) consists in the hot water pre-treatment of biomass at about 90° C. during 30 minutes to 2 hours, preferably 1 hour, followed by cooling to room temperature (20° C.-25° C.), and filtration in order to obtain a solid phase (i.e. pre-treated biomass) to be used in step e).

The optional removal of lipids and/or proteins from the biomass, used in the methods described herein, provides biomass more rich in fibres product for further degradation according to step f). The lipids removal is carried out by methods known in the art, such as mechanical press, organic solvents, or supercritical CO₂. The protein removal is carried out by methods known in the art, such as enzymatic, alcohol, water with salts and/or surfactants, or extrusion.

Some ionic polymers used in the method of the invention for producing the prebiotic composition from biomass consists of anions and a polymeric backbone containing cations as disclosed in WO 2019/058270 A1 incorporated by reference in its entirety. Specifically, the ionic polymer (IP) used in the method of the invention for producing the prebiotic composition from biomass consists of a monomer of formula I

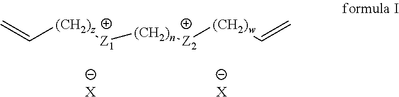

or consists of a first monomer of formula I

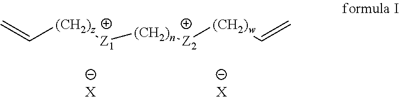

and at least one second monomer selected from the group consisting of

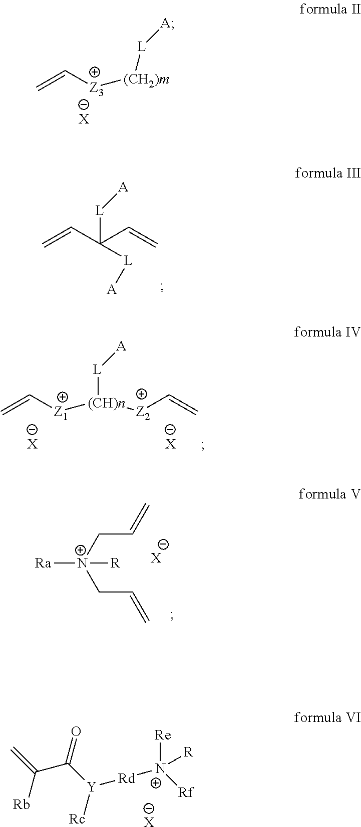

wherein
n and m are independently selected from 1, 2, 3, 4, 5, 6; preferably n and m are independently selected from 1, 2, 3; most preferably n is 2 and m is 1 or 2.
z and w are independently selected from 0, 1, 2, 3; preferably z and w are independently selected from 0 and 1; most preferably z and w are 0 or 1.
Z₁, Z₂ and Z₃ are cations each independently selected from the group comprising:

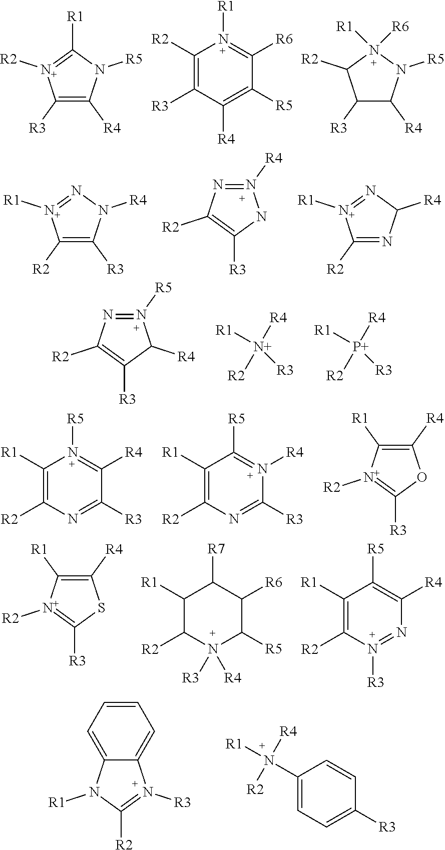

preferably Z₁, Z₂ and Z₃ are cations each independently selected from the group comprising:

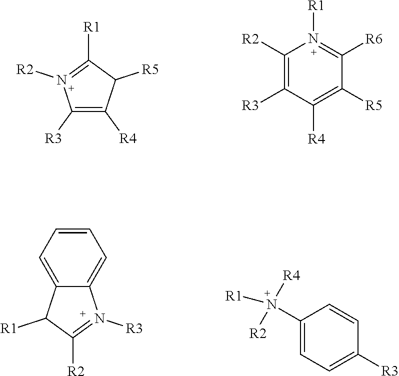

most preferably Z₁, Z₂ and Z₃ are cations each independently selected from the group comprising:

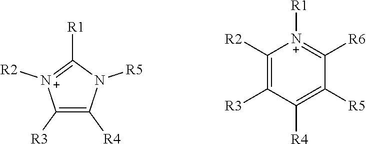

R1, R2, R3, R4, R5, R6 and R7 are each independently selected from the group comprising a bond, H, C₁-C₆ alkyl, C₁-C₆ allyl, —CH₂—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond; preferably R1, R2, R3, R4, R5, R6 and R7 are each independently selected from the group comprising a bond, H, C₁-C₆ alkyl, provided that two of R1, R2, R3, R4, R5, R6 and R7 are each a bond; most preferably R1, R2, R3, R4, R5, R6 and R7 are each independently selected from the group comprising a bond and H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group comprising H, —SO₃H, —COOH, —[P(═O)(OH)₂], —O—SO₃H, —O—COOH, —O—[P(═O)(OH)₂], preferably L is absent;
A is an optional acidic group and each occurrence of A, if present, is independently selected from the group comprising H, —SO₃H, —COOH, —[P(═O)(OH)₂], —O—SO₃H, —O—COOH, —O—[P(═O)(OH)₂], —CH₂—COOH, provided that when z and w are 0, A is present in formula IV; preferably each occurrence of A, if present, is independently selected from the group comprising H, —SO₃H, —COOH, —O—COOH, —CH₂—COOH, provided that when z and w are 0, A is present in formula IV; most preferably A is absent or occurrence of A, if present, is independently selected from the group comprising H, —COOH, —CH₂—COOH, provided that when z and w are 0, A is present in formula IV;
X⁻ is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate; preferably X⁻ is selected from the group comprising F⁻, Cl⁻, HSO₄⁻, SO₄²⁻, PO₄³⁻, HPO₄²⁻, CF₃CO₂⁻, CF₃CO₃⁻, CF₃SO₃⁻; most preferably X⁻ is selected from the group comprising Cl⁻, HSO₄⁻, SO₄²⁻, CF₃SO₃⁻.
Ra is C₁-C₂₄ alkyl;
Rb and Rc are each independently selected from the group comprising H and CH₃ or absent, preferably Rc is absent;
Rd is C₁-C₂₄ alkylene or C₁-C₂₄ alkyl, optionally substituted by C₁-C₂₄ alkyl, preferably Rd is C₁-C₂ alkylene or C₁-C₂ alkyl or C₁-C₃ alkyl;
Re and Rf are each independently C₁-C₂₄ alkyl, preferably CH₃;
Y is N or O, provided that when Y is O, Rc is absent;
R is selected from the group comprising C₁-C₂₄ alkyl and C₅-C₁₀ aryl or is absent; preferably R is CH₃;

In the context of the present invention, the feature “A is an optional acidic group” means that A is a group, preferably an acidic group, that is optional, i.e that A is present or absent.

In some embodiments of the method, the second monomer of formula VI of the ionic polymer (IP) is

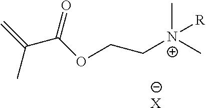

In some embodiments of the method, the second monomer of formula VI of the ionic polymer (IP) is

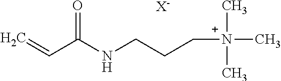

In some embodiments of the method, the (first) monomer of formula I of the ionic polymer (IP) is

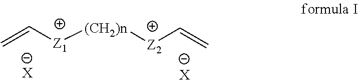

In some embodiments of the ionic polymer of the method of the present invention, Z₁ and Z₂ are same (identical). In other embodiments, Z₁ and Z₂ are different.

In some embodiments of the ionic polymer of the method of the present invention, when Z₁ and Z₂ is

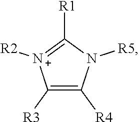

wherein R2 and R5 are bonds and R1, R3 and R4 are H, n is not 4.

In other embodiments of the ionic polymer of the present invention, when Z₁ and Z₂ is

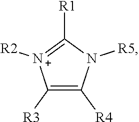

wherein R2 and R5 are bonds and n is 4, at least one of R1, R3 and R4 is not H.

In some preferred embodiments of the ionic polymer of the method of the present invention, C₁-C₆ carboxylate are selected from the group comprising formate, acetate, propionate, butyrate, hexanoate, maleate, fumarate, oxalate, lactate, pyruvate.

The ratio between different monomers in the ionic polymers of the invention that comprises the first monomer and the second monomers can be any suitable ratio and may vary depending on the biomass to be processed. In some embodiments, the first and the second monomers are present in ratio 1:1 or 4:1. In some other embodiments, the ratio between the first and the second monomers used in the methods described herein is ranging from 4:1 to 1:4.

According to some embodiments of the method of the present invention, monomers according to formula I are selected from the group comprising

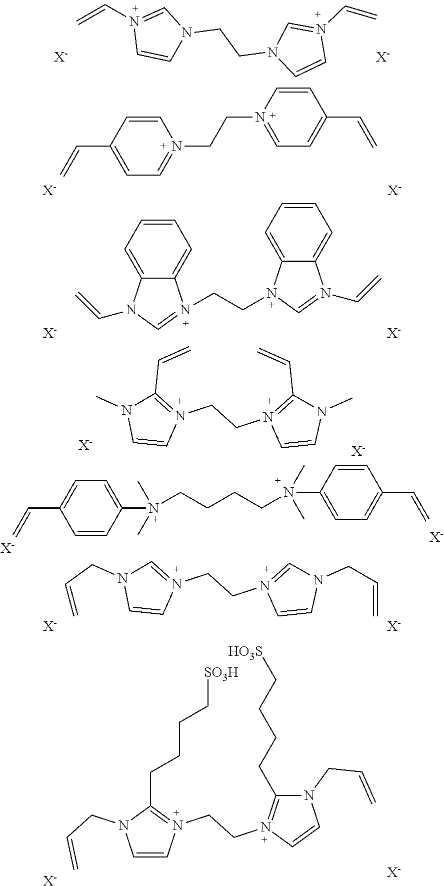

According to further embodiments of the method of the present invention, monomers according to formula I are selected from

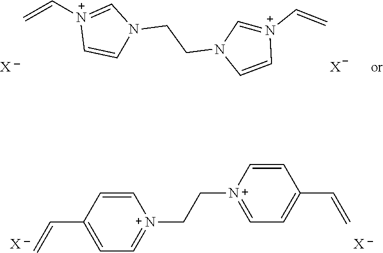

According to some embodiments of the method of the present invention, monomer according to formula II is

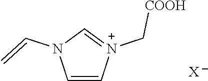

According to some embodiments, the method of the present invention provides ionic polymers selected from the group comprising

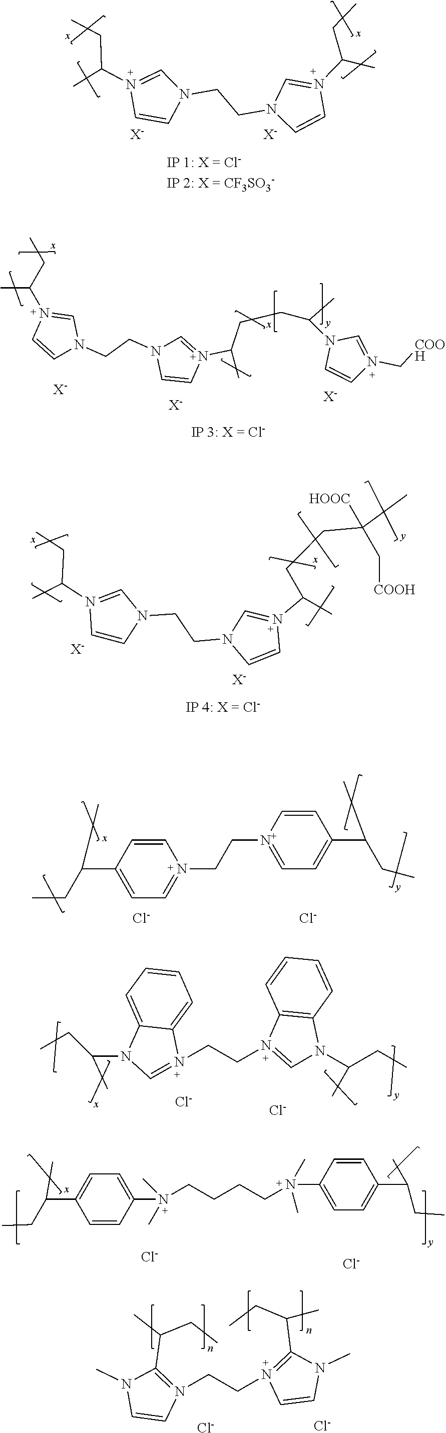

x and y are integers each independently selected within the range 1 to 1000; preferably 1 to 500 or 1 to 200; more preferably 1 to 100 or 1 to 50;

According to some embodiments, the method of the present invention provides ionic polymers selected from the group comprising

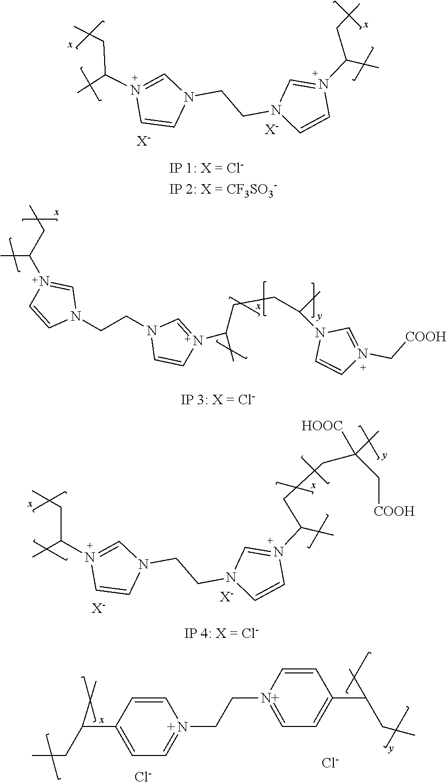

x and y are integers each independently selected within the range 1 to 1000; preferably 1 to 500 or 1 to 200; more preferably 1 to 100 or 1 to 50;

According to an embodiment of the method of the invention, the ionic polymer is

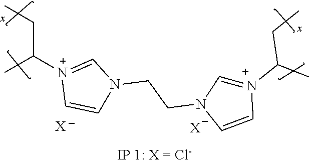

According to another embodiment of the method of the invention, the ionic polymer (IP-A5) consists of the monomer of formula (I) and the monomer of formula VI as follows:

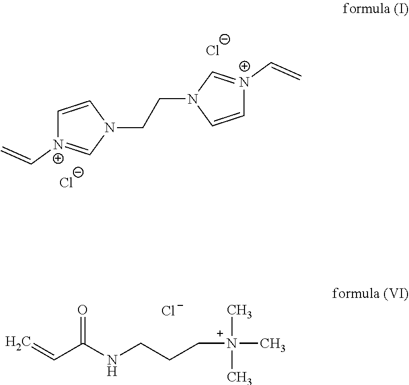

According to another embodiment of the method of the invention, the ionic polymer (IP-A6) consists of the monomer of formula (I) and the monomer of formula (VI) as follows:

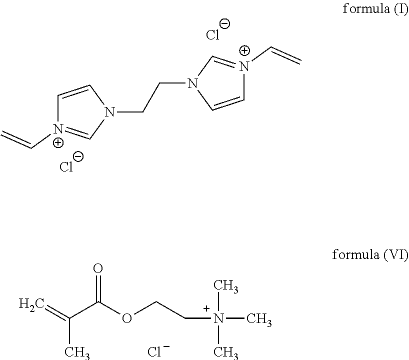

In some embodiments of the method for producing the prebiotic composition, the organic solvent is selected from the group comprising alcohol (such as methanol, ethanol, butanol, ethylene glycol, etc., preferably ethanol), ether (such as dimethoxyethane, diglyme, butyl methyl ether, etc.), ketone (such as methyl isobutyl ketone, N-methyl-2-pyrrolidone, etc.), eutectic solvent (such as glycerol, choline chloride, octanoic acid, tetrabutylammonium chloride, poly (ethylene glycol), choline chloride, lactic acid, glycine).

In some embodiments of the methods of the present invention, recovering the prebiotic composition can be done by any technic known in the art, such as filtration, centrifugation or gravity settling. After the recovering, the prebiotic composition can be used in a liquid form or concentrated or dried to a powder form.

The effective amount of the ionic polymers of the invention or a combination thereof used in the methods described herein can depend on several factors including, for example, the type of the biomass, the amount of the biomass, the content of proteins, carbohydrates and/or lipids in the biomass, the type and number of pre-treatment(s) applied to the biomass, and the reaction conditions (such as temperature and time). An effective amount of the ionic polymer of the invention refers to an amount sufficient to degrade biomass into the prebiotic composition of the invention. In some embodiments, the effective amount of the ionic polymer of the invention is usually 0.005:1 w/w to 10:1 w/w, 0.05:1 w/w to 10:1 w/w, 0.5:1 w/w to 10:1 w/w, 1:1 w/w to 1:5 w/w, preferably 0.1:1 w/w to 1:5 w/w, most preferably 0.005:1 w/w to 0.5:1 w/w, compared to in the biomass loading.

The ratio biomass to water used in the methods described herein can depend on several factors, including for example the type of biomass and the amount of biomass. In some embodiments, the ratio biomass to water and/or organic solvent (such as alcohol, ether, ketone, eutectic solvent) used in the methods described herein is ranging from 1:100 w/v to 1:1 w/v, preferably 1:50 w/v to 1:10 w/v or preferably 0.5:10 w/v to 1.5:10 w/v.

The preferred temperature profile for the heating used in the methods described herein depends on the biomass starting material being used and also the intended monomer and oligomer mixture being produced. The heating temperature should preferably be held at a maximum of 170° C., in some embodiments at a maximum of 150° C. In some embodiments, the heating temperature is between 50° C. and 170° C., or between 80° C. and 170° C. preferably between 100° C. to 150° C. or between 100° C. to 130° C. Preferably, for small-scale applications, the heating is done in a high-pressure autoclave reactor, which after sealing, is heated for appropriate reaction time and temperature.

In some embodiments, the appropriate reaction time in the methods described herein is for example between 10 minutes and 10 hours, preferably between 0.5 hour and 5 hours, or 0.5 hours and 3 hours, or between 1 hour and 3 hours, depending on the type and amount of biomass.

The method for producing the prebiotic composition operates at moderate temperatures, typically less than 150° C., whereas the prior art methods need temperatures of more than 150° C. In addition, the method for producing the prebiotic compositions of the invention provides fewer by-products, which allows easier recovery of the desired products.

Another aspect of the present invention provides a prebiotic composition obtained by the method of the invention for producing the prebiotic composition.

In various embodiments, the prebiotic composition of the invention includes at least one carbohydrate compound. Such a carbohydrate compound can be a bio-based compound. A benefit of a carbohydrate compound can be improving the balance of a body area commensal microbiota, by providing an energy source to promote beneficial microbial growth in the body area. In some embodiments, the at least one carbohydrate compound includes an inulin, an alpha-glucan oligosaccharide, a fructooligosaccharide, an isomaltooligosaccharide, an xylooligosaccharide, an arabinoxylo-oligosaccharide, a beta-glucan, a transgalactooligosaccharide, mannan-oligosaccharide, lactulose, xylitol, lactitol, trehalose, or combinations thereof. The prebiotic composition of the invention can further include hydrolysed proteins and phenolics (such as ferulic acid).

In some embodiments, the prebiotic composition of the invention comprises (in wt %):
Total oligosaccharides: 50% to 70%
Proteins and/or protein hydrolysates: 20% to 40%
Phenolic compounds (Phenolics): <1%
Minerals: <8%
Moisture: <10%
   wherein the oligosaccharides are selected from the group comprising
Glucose: 1% to 20%, by weight of total oligosaccharides
Xylose: 20% to 40%, by weight of total oligosaccharides
Arabinose: 5% to 30%, by weight of total oligosaccharides
   and wherein distribution of degree of polymerisation (DP) of said oligosaccharides is
DP1: 0% to 10%
DP2 to DP30: >80%.

A particular embodiment provides the prebiotic composition of the invention, wherein oligosaccharides comprise β-(1,4)-linked xylose units, β-(1,4)-linked xylose units substituted with α-(1,2) arabinose or α-(1,3) arabinose or α-(1,2) arabinose and α-(1,3) arabinose together, linked glucose units forming (1,3)-(1,4)-β-D-glucan molecules.

Another particular embodiment provides the prebiotic composition of the invention, wherein oligosaccharides comprise arabinoxylo-oligosaccharides, both linear and branched in their structure, preferably with at least one arabinose or xylose positioned at the reducing end of the oligosaccharide backbone. The ratio between arabinose and xylose units in oligosaccharides varies depending on the reaction conditions and ionic polymer applied. Thus in one embodiment, in the prebiotic composition of the invention the branched oligosaccharides comprise arabinose and xylose units, preferably with an arabinose/xylose ratio of 0.4-1.2, preferably a ratio of 0.4-1.0, preferably a ratio of 0.45-1.0, preferably a ratio of 0.5-0.9. In one embodiment of the prebiotic composition, the branched or linear oligosaccharides comprise xylose units and practically free of arabinosyl groups.

In one embodiment of the prebiotic composition of the invention, the oligosaccharides comprise at least two β(1-4) glucose unit linked together.

In one embodiment of the prebiotic composition of the invention, the oligosaccharides comprise at least two β(1-4) linked xylose unit linked together.

In one embodiment of the prebiotic composition of the invention, the oligosaccharides comprise at least one branch consisting of α (1-3) linked arabinose unit or β(1-3) linked xylose unit linked to the backbone.

In one embodiment of the prebiotic composition of the invention, the oligosaccharides comprise β(1-4) linked xylose units are linked with at least one α(1-2) arabinose unit.

In one embodiment of the prebiotic composition of the invention, the oligosaccharides comprise β(1-4) linked xylose units where monosubstituted xyloses linked with at least one a (1-2) arabinose unit and one α (1-3) arabinose unit.

In one embodiment of the prebiotic composition of the invention, the oligosaccharides comprise β(1-4) linked xylose units where at least one disubstituted xylose linked with one α(1-2) arabinose unit and one α(1-3) arabinose unit.

In one embodiment of the prebiotic composition of the invention, the oligosaccharides comprise β(1-4) linked xylose units where at least one monosubstituted xylose with α(1-3) arabinose unit and disubstituted xylose with one α(1-2) arabinose unit and one α(1-3) arabinose exist.

In one embodiment of the prebiotic composition of the invention, the oligosaccharides comprise at least one trisaccharide, wherein xylose is linked with α(1-4) linkage between two glucose units (Glc α(1-4) Xyl α(1-4) Glc) or one arabinose and xylose unit (Ara α(1-6) [Xyl β(1-4)] Glc).

In one embodiment of the prebiotic composition of the invention, the oligosaccharides comprise at least one branch consisting of α(1-3) linked arabinose unit or β(1-2) linked xylose unit linked to the backbone.

In one embodiment of the prebiotic composition of the invention, the oligosaccharides comprise at least one trisaccharide, wherein xylose is linked with α(1-4) linkage between two glucose units (Glc α(1-4) Xyl α(1-4) Glc) or one arabinose and xylose unit (Ara α(1-6) [Xyl β(1-4)] Glc).

In some embodiments, the backbone of the prebiotic composition of the invention is composed of β-(1,4)-linked xylose residues. It can be substituted with arabinose residues while hydroxycinnamic, ferulic and p-coumaric acids can be esterified on the arabinose residue.

In some embodiments, the backbone of the prebiotic composition of the invention is composed of β-(1,4)-linked xylose residues, which can be substituted with arabinose residues while additional uronic acid or methylated uronic acid may be present on it.

In some embodiments, the oligosaccharides and/or the proteins in the prebiotic composition of the invention have an average molecular weight less than 10 kDa, preferably less than 7.5 kDa, preferably less than 5 kDa, preferably less than 4 kDa, preferably less than 2 kDa.

One advantage of the prebiotic composition of the invention is that it provides no turbidity when it is used in cosmetic compositions. Another advantage of the prebiotic composition of the invention is that it has almost no colour, or very light colour, which does not color the cosmetic composition.

Another aspect of the present invention provides a topical cosmetic composition comprising:
an effective amount of the prebiotic composition of the invention, and
at least one dermatologically acceptable carrier.

The topical cosmetic compositions herein include an effective amount of the prebiotic composition of the invention. The prebiotic composition may be present at an amount of greater than 0.001%, 0.01%, 0.05%, 0.1%, 0.5%, 1%, 2%, 3%, 4% or even greater than 5% by weight of the topical cosmetic composition. It may be desirable to limit the amount of the prebiotic composition in the present topical cosmetic compositions to an amount of less than 25%, 20%, 15%, 10% or even 5% by weight of the composition to avoid cosmetically undesirable characteristics (e.g., stickiness or poor spreadability). In certain embodiments, the prebiotic composition may be present at an amount 0.1% to 10%, preferably 0.5% to 5% by weight of the topical cosmetic composition, most preferably 0.001% to 5% by weight of the topical cosmetic composition.

In certain embodiments, the prebiotic composition may be present at an amount sufficient to increase the bacterial counts of at least one skin commensal microorganism by at least 10% in vitro (e.g., from 10-200% or more, 50-175%, 100-150%, or any value in these ranges). Additionally or alternatively, the prebiotic composition may be present at an amount sufficient to increase the bacterial counts of at least two skin commensal microorganisms by at least 10% in vitro (e.g., from 10-200%, 20-180%, 30-160%, 40-150%, 50-120%, or any value in these ranges). Further, the prebiotic composition may be present at an amount sufficient to increase the bacterial counts of at least three skin commensal microorganisms by at least 10% in vitro (e.g., from 10-200% or more or any value in this range). The in vitro bacterial counts may be determined according to the Plate Count Test described in more detail below.

The topical cosmetic compositions of the invention include one or more dermatologically acceptable carriers. Such carriers can be selected from the group comprising:
Water and/or water miscible solvents. Suitable water miscible solvents include monohydric alcohols, dihydric alcohols, polyhydric alcohols, glycerol, glycols, polyalkylene glycols such as polyethylene glycol, and mixtures thereof. When the topical cosmetic composition is in the form of an emulsion, the water and/or water miscible solvents are typically associated with the aqueous phase of the emulsion.
One or more suitable oils. The oils may be volatile or nonvolatile oils. Volatile oils suitable for use herein may have a viscosity ranging from 0.5 to 5 centistokes (cSt) at 25° C. Volatile oils may be used to promote more rapid drying of the skin care composition after it is applied to skin. Nonvolatile oils may be included to provide emolliency and protective benefits to the skin.
One or more suitable silicone oils such as, for example, one or more polysiloxanes, and/or hydrocarbon oils (e.g., straight, branched, or cyclic alkanes and alkenes) and/or amides (suitable amides include N-acetyl-N-butylaminopropionate, isopropyl N-lauroylsarcosinate, and N,N, -diethyltoluamide), and/or ethers (suitable examples of these ethers include PPG-14 butyl ether, PPG-15 stearyl ether, dioctyl ether, dodecyl octyl ether, and mixtures thereof).
Emulsifier. An emulsifier may be desirable when the composition is provided in the form of an emulsion or if immiscible materials are being combined.
Structuring agents may be used to increase viscosity, thicken, solidify, or provide solid or crystalline structure to the topical cosmetic composition. Structuring agents are typically grouped based on solubility, dispersibility, or phase compatibility. Examples of aqueous or water structuring agents include polymeric agents, natural or synthetic gums, polysaccharides, and the like. Other exemplary classes of polymeric structuring agents include but are not limited to carboxylic acid polymers, polyacrylamide polymers, sulfonated polymers, high molecular weight polyalkylglycols or poly glycerins, copolymers thereof, hydrophobically modified derivatives thereof, and mixtures thereof.

The topical cosmetic compositions of the invention may optionally include ingredients useful for regulating and/or improving a condition of mammalian skin. Some non-limiting examples of such optional ingredients include vitamins (one or more water-soluble vitamins, such as vitamin B, vitamin C, vitamin D, vitamin K); peptides and peptide derivatives; sugar amines, sunscreen actives (or sunscreen agents) and/or ultraviolet light absorbers, humectant, moisturizer, skin conditioner, colorants (pigments, dyes, lakes, combinations of these and the like), film-forming compositions, phytosterols, salicylic acid compounds, hexamidines, dialkanoyl hydroxyproline compounds, flavonoids, retinoid compounds, botanicals, N-acyl amino acid compounds, their derivatives, and combinations thereof.

The topical cosmetic compositions of the invention may be in any one of a variety of forms known in the art, including, for example, an emulsion, lotion, milk, liquid, solid, cream, gel, mouse, ointment, paste, serum, stick, spray, tonic, aerosol, foam, pencil, and the like. The cosmetic compositions may also be incorporated into shave prep products, including, for example, gels, foams, lotions, and creams, and include both aerosol and non-aerosol versions. Other cosmetic compositions include antiperspirant, deodorant, and personal cleaning compositions such as soap and shampoo.

The topical cosmetic compositions of the invention may be prepared according to conventional methods known in the art for making such compositions. Such methods may include mixing ingredients in one or more steps to achieve a relatively uniform state, with or without heating, cooling, application of vacuum, and the like. For example, emulsions may be prepared by first mixing the aqueous phase materials separately from the fatty phase materials and then combining the two phases as appropriate to yield the desired continuous phase. In certain embodiments, the compositions may be prepared to provide suitable stability (physical stability, chemical stability, photostability, etc.) and/or delivery of active materials. The composition may be provided in a package sized to store a sufficient amount of the composition for a treatment period. The size, shape, and design of the package may vary widely.

Another aspect of the present invention provides use of the prebiotic composition of the invention or the topical composition of the invention for non-therapeutic treatment of the skin. In some embodiments of the invention, the non-therapeutic treatment is protection and/or regeneration of the skin. In other embodiments of the invention, the non-therapeutic treatment is protecting and improving the condition and/or appearance of the skin. In other embodiments of the invention, the non-therapeutic treatment is maintaining skin hydration. In other preferred embodiments, the non-therapeutic treatment is regeneration and/or protection of the skin, improving the condition and/or appearance of the skin and is maintaining skin hydration.

In some embodiments, the present invention provides a method for non-therapeutic treatment of the skin, the method comprising applying an effective amount of the prebiotic composition of the invention or the topical composition of the invention to the skin. In preferred embodiments, the non-therapeutic treatment is regeneration and/or protection of the skin. In other preferred embodiments, the non-therapeutic treatment is improving the condition and/or appearance of the skin. In other preferred embodiments of the invention, the non-therapeutic treatment is maintaining skin hydration. In other preferred embodiments, the non-therapeutic treatment is regeneration and/or protection of the skin, improving the condition and/or appearance of the skin and is maintaining skin hydration.

Another aspect of the present invention provides use of the prebiotic composition of the invention or the topical composition of the invention for modulating the growth of at least one commensal bacterium (microorganism) of the skin. In preferred embodiments, modulating is promoting or enhancing the growth of at least one commensal bacterium. In some embodiments, the commensal bacterium is selected from the group consisting of Propionibacterium acnes, Corynebacterium tuberculostearicum, Streptococcus mitis, Streptococcus oralis, Streptococcus pseudopneumoniae, Streptococcus sanguinis, Micrococcus luteus, Staphylococcus epidermidis, Staphylococcus capitis, Veillonella parvula. In a preferred embodiment, the commensal bacterium is Staphylococcus epidermidis.

Another aspect of the present invention provides a method for modulating the growth of at least one commensal bacterium (microorganism) of the skin, the method comprising applying an effective amount of the prebiotic composition of the invention or the topical composition of the invention to the skin. In some embodiments, the commensal bacterium is selected from the group consisting of Propionibacterium acnes, Corynebacterium tuberculostearicum, Streptococcus mitis, Streptococcus oralis, Streptococcus pseudopneumoniae, Streptococcus sanguinis, Micrococcus luteus, Staphylococcus epidermidis, Staphylococcus capitis, Veillonella parvula. In a preferred embodiment, the commensal bacterium is Staphylococcus epidermidis.

Another aspect of the present invention provides use of the prebiotic composition of the invention or the topical composition of the invention for preventing dysbiosis. In preferred embodiments, the present invention provides use of the prebiotic composition of the invention or the topical composition of the invention for preventing dysbiosis on an external surface of a human or animal body, preferably on external part of human or animal skin. In some embodiments, such use of the prebiotic composition of the invention or the topical composition of the invention is a non-therapeutical use.

Another aspect of the present invention provides a method for preventing dysbiosis, the method comprising applying an effective amount of the prebiotic composition of the invention or the topical composition of the invention to the skin. In a preferred embodiment, the present invention provides a method for preventing dysbiosis on an external surface of a human or animal body, preferably on external part of human or animal skin, the method comprising applying an effective amount of the prebiotic composition of the invention or the topical composition of the invention to the skin. In some embodiments, such use of the prebiotic composition of the invention or the topical composition of the invention is a non-therapeutical use.

Dysbiosis typically involves the loss of beneficial bacterium (microorganisms), such as commensal bacterium, and an expansion of pathogenic bacterium (microorganisms).

Another aspect of the present invention provides use of the prebiotic composition of the invention or the topical composition of the invention for stimulating immune skin system. In preferred embodiment, the stimulation of immune skin system is obtained via increasing the growth and/or colonization of S. epidermidis on the skin or commensal bacteria (microorganisms) on the skin. In some embodiments, stimulating immune skin system provides enhanced immunity to an external surface of a human or animal body, such as human or animal skin. In some embodiments, such use of the prebiotic composition of the invention or the topical composition of the invention is a non-therapeutical use.

Another aspect of the present invention provides a method for stimulating immune skin system, the method comprising applying an effective amount of the prebiotic composition of the invention or the topical composition of the invention to the skin. In preferred embodiment, the stimulation of immune skin system is obtained via increasing the growth and/or colonization of S. epidermidis on the skin or commensal bacteria (microorganisms) on the skin. In some embodiments, stimulating immune skin system provides enhanced immunity to an external surface of a human or animal body, such as human or animal skin. In some embodiments, such use of the prebiotic composition of the invention or the topical composition of the invention is a non-therapeutical use.

The prebiotic composition of the invention is capable of being selectively utilized by microorganisms (bacteria) on and/or in the skin to provide a health benefit, such as protection and/or regeneration of the skin and/or protecting and improving the condition and/or appearance of the skin. Such a prebiotic composition can provide a health benefit of selective stimulation of beneficial commensal microorganisms, competitively against and to the detriment of the growth of opportunistic, undesirable, or pathogenic microorganisms. Such a health benefit can include helping commensal and beneficial microorganisms to flourish on and/or in the skin. For example, in some embodiments, the prebiotic composition of the invention may promote the growth of the commensal bacterium Staphylococcus epidermidis (S. epidermidis) and/or other commensal bacteria. A health benefit can include protecting and promoting the balance and diversity of the microbiota on and/or in a skin. Another health benefit can include promoting, restoring, reinforcing and maintaining skin defence against excessive growth or infection by harmful microbes, or against external toxic substances. Such a health benefit can include the stimulation of antimicrobial peptide release by keratinocytes in the skin. Another health benefit can include increasing the skin's resistance to pathological conditions, including but not limited to dermatitis, acne, rosacea, eczema, and premature aging. Further, such a prebiotic composition can improve the conditions and/or appearance of the skin.

In some embodiments, by variation of parameters of the method of the present invention for producing the prebiotic composition, it is possible to obtain the prebiotic composition that has only prebiotic effect or to obtain the prebiotic composition that has both prebiotic effect and antipathogen effect. Antipathogen effect means that the composition inhibits growths of pathogenic bacteria.

Colonization of S. epidermidis in the skin microbiome may affect the immune function of the skin and/or reduce premature aging of the skin. S. epidermidis promotes restoration of healthy skin, in part, by preventing colonization of the skin microbiome with pathogenic microbes. S. epidermidis has also been shown to influence host immunity by boosting the host immunity against S. aureus, activating mast cell-mediated immunity, suppressing uncontrolled inflammatory reactions during wound healing, inducing skin's production of antimicrobial proteins, and stimulating cutaneous T-cell maturation. Accordingly, S. epidermidis may work in cooperation with the host defence system and endogenous AMPs to protect the skin. Moreover, the microbiome may represent a kind of filter for the environment as most agents in contact with and/or penetrating through the skin are also in contact with the microbiota.

In other embodiment, the prebiotic composition of the invention applied to the skin may increase the colonization of S. epidermidis on the skin, resulting in protecting the skin from premature aging and reducing signs of aging such as reducing hyperpigmentation, wrinkles, and inflammation and improving the adaptive capability of the skin. The skin microbiome modulating composition may also protect the skin from oxidative damage from the environment. Oxidative damage from ultraviolet irradiation from the sun and air pollution may cause deleterious effects in human skin, including sunburn, immune suppression, and premature aging such as photoaging which may be characterized in part by wrinkles, altered pigmentation, and loss of skin tone.

It is believed, without being limited by theory, that the health of the skin microbiome may be linked to desirable skin function or appearance and/or may otherwise provide one or more skin care benefits. For example, it may be possible to maintain or improve the appearance, barrier function, moisture retention and/or other properties of skin by maintaining or improving the health of one or more members of the skin microbiome. In some instances, if a particular area or areas of the skin exhibit undesirable function and/or appearance it may be desirable to target that particular area or areas of the skin for maintenance or improvement. For example, it may be desirable to target particular areas of the skin such as on the face (e.g., forehead, cheeks, and peri-orbital portions of the face), hands and/or forearms, which tend to be more damaged by exposure to the environment (e.g., UV radiation, wind, pollution, oxidation, irritants) than some other areas of the skin and/or which may be subject to visible signs of intrinsic aging.

The prebiotic compositions or the topical cosmetic compositions disclosed herein may be suitable for use as topical skin care or cosmetic products, which may be applied as part of a user's routine makeup or personal care regimen. Additionally or alternatively, the prebiotic compositions or the topical cosmetic compositions herein may be used on an “as needed” basis. In certain embodiments, a skin care product such as a moisturizing cream, lotion or ointment that includes at least one dermatologically acceptable carrier and an effective amount of the prebiotic composition of the invention may be topically applied to one or more target areas of a user's skin (e.g., face, forearms, hands or portions of these) to provide a skin care benefit or otherwise improve the health and/or appearance of the skin in the target area(s). In certain embodiments, the prebiotic composition of the invention may be incorporated into a color cosmetic product such as a foundation that is applied to a user's face or portions thereof as part of a daily beauty regimen.

In certain embodiments, particular areas of the skin may be identified as being in need of a skin care benefit that can be addressed through the use of the prebiotic composition of the invention or the topical cosmetic compositions of the invention. For example, areas of the face (e.g., nose, cheeks, forehead, chin, around the eyes), the front and back of the neck, the top of a hand, the top of a forearm, the shoulders and/or a major body fold may be identified as being in need of treatment by the prebiotic compositions or the topical cosmetic compositions of the invention. Of course, it is to be appreciated that the prebiotic compositions or the topical cosmetic compositions disclosed herein may be applied to any portion of skin on the body (e.g., feet, legs, back, upper arm, torso, buttocks) to provide a cosmetic benefit, and such portions of the skin may be identified as target areas.

The topical cosmetic compositions of the invention may be applied one or more times per day as part of a user's regular beauty regimen (e.g., showering, applying makeup, applying moisturizers or other skin care or hair care products). The topical cosmetic compositions of the invention may be applied more than once per day, for example, once at the beginning of the day, once in the middle of the day, and/or once at the end of the day. In some instances, the topical cosmetic compositions may be applied whenever a user applies or reapplies other cosmetic compositions such as lipstick or mascara. In some instances, it may be desirable to apply the topical cosmetic compositions of the invention every other day, two or three times per week, weekly, biweekly or monthly, as desired. It may be desirable to apply the topical cosmetic compositions of the invention such that at least a portion of the composition (e.g., the prebiotic portion) is present on the user's skin for at least an hour (e.g, from 1 to 24 hours, from 2 to 20 hours, from 4 to 16 hours, or from 8-12 hours). In certain embodiments, it may be desirable to apply the composition such that at least a portion of the composition is present on skin for more than a day (e.g., 1-7 days, 2-6 days, 3-5 days, or even 4 days). In certain embodiments, it may be desirable to apply the topical cosmetic compositions of the invention at one or more of the foregoing frequencies for at least two consecutive or non-consecutive application periods. For example, the composition may be applied once per day for 2, 3, 4, 5, 6, or 7 consecutive or non-consecutive days. In another example, the present cosmetic composition may be applied every other day for a month or more.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### EXAMPLES

### Preparation of Brewers' Spent Grains (BSG) Extracts

### Preparation of BSG-Derived P1

500 g of brewers' spent grains (BSG) (dry bases) suspended in 3.75 L of water together with ionic polymer catalyst “IP1” were stirred at 140° C. for 1 h. After reaction, the mixture was cooled to room temperature, filtered and the liquid phase was dried using spray drier. The obtained dried powdered product was analysed and used for activity study. P1 contained 57.9% of oligosaccharides, with 44.7% and 13.2% as arabinoxylan and glucan respectively. See Table 1.

### Preparation of BSG-Derived P2

267 g of BSG (dry bases) suspended in 4 L of water were stirred at 90° C. for 1 h. Afterwards, the mixture was cooled to room temperature, filtered and solid phase was resuspended in 2.850 L of water together with “IP1” and stirred at 140° C. for 1 h. After reaction, the mixture was cooled to room temperature, filtered and the liquid phase was dried using spray drier. The obtained dried powdered product P2 was analysed and used for activity study. P2 contained 62% of oligosaccharides, with 51% and 11% as arabinoxylan and glucan respectively. See Table 1.

### Preparation of BSG-Derived P3

200 g of BSG (dry bases) suspended in 1.5 L of water and 100 ml of ethanol together with ionic polymer catalyst “IP1” were stirred at 145° C. for 1.5 h. After reaction, the mixture was cooled to room temperature, filtered and the liquid phase was dried using spray drier. The obtained dried powdered product was analysed and used for activity study. P3 contained 53.5% of oligosaccharides, with 38.3% and 15.2% as arabinoxylan and glucan respectively. See Table 1.

### Preparation of BSG-Derived P4

200 g of BSG (dry bases) suspended in 1.5 L of water together with ionic polymer catalyst “IP1” were stirred at 140° C. for 1 h. After reaction, the mixture was cooled to room temperature and filtered. Certain amount of bentonite was added to the filtrate and the mixture was stirred at RT for 1 h. Afterwards, the mixture was filtered and the liquid phase was dried using spray drier. The obtained dried powdered product was analysed and used for activity study. P4 contained 57% of oligosaccharides, with 41.4% and 15.6% as arabinoxylan and glucan respectively. See Table 1.

### Preparation of BSG-Derived P5

500 g of BSG (dry bases) suspended in 3.75 L of water together with ionic polymer catalyst “IP1” were stirred at 140° C. for 1 h. After reaction, the mixture was cooled to room temperature, filtered and the solid leftover fraction in presence of catalyst was redispersed in water again and one more hydrolysis cycle was performed. After cooling, the same type of manipulations was performed, and the final hydrolysis cycle was completed. After cooling, the obtained mixture was filtered, and the liquid phase was dried using spray drier. The obtained dried powdered product was analysed and used for activity study. P5 contained 52.9% of oligosaccharides, with 48% and 4.9% as arabinoxylan and glucan respectively. See Table 1.

TABLE 1

| g/100 g of product (a prebiotic | | | | | |
|---|---|---|---|---|---|
| composition) | P1 | P2 | P3 | P4 | P5 |
| Total oligosaccharides, | 57.9 | 62.5 | 53.5 | 57 | 52.9 |
| of which: | | | | | |
| Glucose | 13.2 | 11.1 | 15.2 | 15.6 | 4.9 |
| Arabinose | 15.5 | 21.1 | 11.1 | 16.8 | 9.8 |
| Xylose | 29.2 | 30.3 | 27.2 | 24.6 | 38.2 |
| Protein | 23 | 22 | 23.7 | 21 | 34.6 |
| Minerals | 5.4 | 5.4 | 5.7 | 5.8 | 7.1 |
| Phenolics | <1 | <1 | <1 | <1 | <1 |
| Moisture | 7 | 7 | 7 | 7 | 7 |

### Preparation of BSG-Derived P6

The ionic polymer IP-A5 was prepared, that consists of the monomer of formula (I) and the monomer of formula VI as follows:

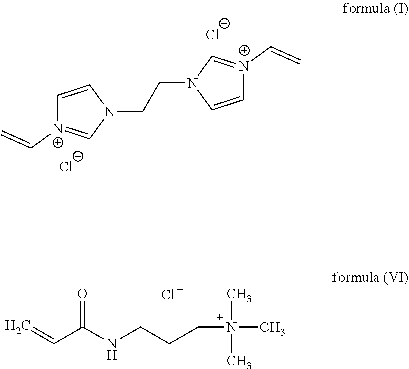

IP-A5 was used for spent barley hydrolysis.

500 g of brewers' spent grains (BSG) (dry bases) suspended in 3.75 L of water together with ionic polymer catalyst “IP-A5” were stirred at 130° C. for 1.5 h. After reaction, the mixture was cooled to room temperature, filtered and the liquid phase was dried using spray drier. The obtained product contained 40.3% of oligosaccharides, with 35.7% and 4.6% as arabinoxylan and glucan respectively.

### Preparation of BSG-Derived P7

The ionic polymer IP-A6 was prepared, that consists of the monomer of formula (I) and the monomer of formula (VI) as follows:

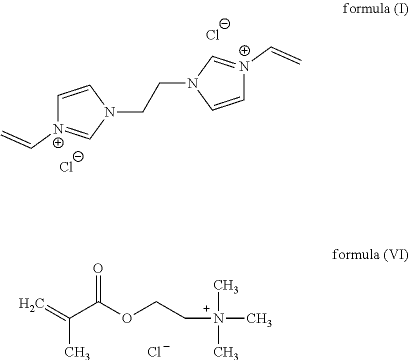

IP-A6 was used for spent barley hydrolysis.

500 g of brewers' spent grains (BSG) (dry bases) suspended in 3.75 L of water together with ionic polymer catalyst “IP-A6” were stirred at 130° C. for 1.5 h. After reaction, the mixture was cooled to room temperature, filtered and the liquid phase was dried using spray drier. The obtained product contained 61.5% of oligosaccharides, with 46.8% and 14.7% as arabinoxylan and glucan respectively.

### Activity Study

A microbiological screening study on liquid broth was performed to assess the activity (bacterial growth) of 5 preparations (P1, P2, P3, P4 and P5) on two Staphylococcus species: (1) Staphylococcus aureus (MRSA) ATCC 33591 as potential skin pathogen and (2) Staphylococcus epidermidis ATCC 12228 as commensal strain in healthy skin. With the aim to assess a potential and strain specific antimicrobial or prebiotic activity vs Staphylococcus spp and to validate the hypothesis that the ingredients may have a prebiotic effect (on S. epidermidis) and meanwhile negatively impacting the viability or growth rate (on S. aureus), the experimental results are presented below.

### Prebiotic Validation

To assess a potential and strain specific antimicrobial or prebiotic activity vs Staphylococcus spp.: a microdilution assay (viable count on agar plates) in liquid broth was performed using 3 different concentrations, 4%-2%-1% for each ingredient, compared to inulin and glucose as carbon source and prebiotic reference.

Each strain was inoculated in a medium poor in nutrients added with 3 concentrations of the test item and let grow up to 24 h; then the bacterial viability measurement at 4 h and 24 h, corresponding to early exponential and stationary phase respectively, was performed by means a viable count on agar plate (CFU/mL), to evaluate the interference on bacterial growth. Each treatment was performed in triplicate.

The growth rate checked by viable count (CFU/mL) was performed to quantify the number of residual viable cells present in the test cultures and they were then expressed as Log values.

See results in Table 2.

The Negative control (NC, culture medium alone) and positive controls PC (Inulin 4% and Glucose 4%) up to 24 h have shown a growth similar to the inoculum count confirming the bacterial vitality and validating the test system.

For the tested BSG-derived ingredients the observed results reveal the following:
bold (in S. epidermidis section) indicates the results when the number of CFUs are higher than both glucose and inulin at 4% (carbon source as prebiotic reference);
light grey indicates the results when the difference between the number of CFUs obtained in presence of the test items (P2 at 1% and P3 at 2%) compared with the CFUs obtained in presence of glucose (4%) or inulin (4%) is more than 0.5 Log (test item higher than controls) that is considered the minimum significant increasement to define a prebiotic effect (bacterial growth).

A minor effect was observed at 24 hours but this could be linked to the high concentration of the bacteria and to the possible achievement of stationary phase in the bacterial growth. It should be noted that bacterial viability measured at 4 h and 24 h corresponds to early exponential and stationary phase, respectively. The Stationary phase (idiophase) is the phase where the microorganism stops its growth, often due to a growth-limiting factor such as the depletion of an essential nutrient; the stationary phase, results so from a situation in which growth rate and death rate are equal (the number of new cells created is limited by the growth factor and as a result the rate of cell growth matches the rate of cell death). Over this phase could be started a decline phase, where bacteria die by lack of nutrients that makes the interpretation at 24 h not very robust in term of the viable growth quantification.

All BSG-derived preparations (P1-P2-P3-P4-P5) have shown a capability to maintain under control the S. aureus growth (bacteriostatic effect). In particular these results indicate the following: -bold (in S. aureus section) indicates the results when the number of CFUs are lower than both glucose and inulin at 4% (carbon source as prebiotic reference)
bold italic indicates the results when the difference between the number of CFUs obtained in presence of the test items compared with the CFUs obtained in presence of glucose (4%) or inulin (4%) is more than 0.5 Log (test item lower than controls) that is considered the minimum significant reduction to define an anti-microbial effect (bacterial growth decrease). A better anti-microbial effect were observed up to 4 hours as during 24 hours only the items P3 (all concentrations) and P2 and P1 at 4% have shown a significant antimicrobial effect vs controls.

### Antipathogen Validation

A next series of experiments were conducted aiming to validate the hypothesis that the ingredients may have a different effect on bacterial species, in particular they can have a prebiotic effect on S. epidermidis and meanwhile negatively impacting the viability or growth rate on S. aureus.

Ingredients were diluted into the minimal growth medium at the 3 required concentrations, added to a mix (ration 1:1) of the two Staphylococcus spp. with a low level of inoculum (102) and let grow up to 24 h; then each bacterial viability measurement at 4 h and 24 h, were performed by means viable count on Chromoselect agar plate (able to differentiate the 2 strains) (CFU/mL), to evaluate the effect on bacterial growth.

See results in Table 3.

The Negative control (NC, culture medium alone) up to 4 h shown a growth not different from the inoculum count confirming the bacterial viability and the test system validity.

All preparations (P1-P2-P3-P4-P5-A82), compared to the controls have shown a capability to maintain under control up to 4 h the bacterial growths (bacteriostatic effect). In particular, the results indicate the following:
bold (in S. epidermidis section) indicates the results when the number of CFUs are higher than the negative control (culture medium alone)
light grey indicates the results when the difference between the number of CFUs obtained in presence of the test items (P5 at 2% after 2 h and P4 at 4% after 4 h) compared with the CFUs obtained in the negative control is more than 0.5 Log (test item higher than control) that is considered the minimum significant increasement to define a prebiotic effect (bacterial growth).
bold (in S. aureus section) indicates the results when the number of CFUs are lower than the negative control (culture medium alone)
bold italic indicates the results when the difference between the number of CFUs obtained in presence of the test items (P3 at all concentrations after 2 h and P1, P2, P3 and P4 higher concentrated, 4%, after 4 h) compared with the CFUs obtained in the negative control is more than 0.5 Log (test item lower than control) that is considered the minimum significant reduction to define an anti-microbial effect (bacterial growth decrease). Considering both effects combined, prebiotic (growth increase) for S. epidermidis (increase of 0.71 Log) and anti-microbial (growth decrease) for S. aureus (reduction of 0.56 Log), it can be concluded that P4 at 4% after 4 hours of contact demonstrated the best results.

### Conclusion

A potential antimicrobial activity vs S. aureus and prebiotic effect vs S. epidermidis: Considering a CFU difference more than 0.5 Log versus both the controls (Inulin and Glucose at 4%) as significant to define the prebiotic (bacterial growth increase) or antimicrobial effect (bacterial growth reduction) the best and robust results were shown by:
P2 at a concentration of 1% after 4 h of contact
P3 at a concentration of 2% after 4 h of contact.

Considering a difference more than 0.5 Log versus the negative control as significant to define the prebiotic (bacterial growth increase) or antimicrobial effect (bacterial growth reduction) the better results with the two combined effects were shown by P4 at the concentration of 4% after 4 hours of contact.

### Human Reconstituted Epidermis (RHE) Tests

The principal aim of the present study was to evaluate the potential efficacy of BSG-derived preparations on a more biologically relevant in vitro preclinical model based on colonized Reconstructed Human Epidermis (RHE). The main focus was given to:
I. to counteract Staphylococcus aureus adhesion and its proliferative capacity. The rationale was based on the possibility of the ingredients to balance the inflammatory reaction induced by the bacterium, reducing its adhesion and growth on RHE model (inhibitory study);
II. the prebiotic effect of the 3 preparations versus Staphylococcus epidermidis (prebiotic study). The rationale was based on the possibility of the ingredients to protect and biostimulant the normal skin microbiota;
III. their biological impact on sterile RHE model. The rationale was based on the possibility that the ingredients to enhance or balance the innate immune response.

For this study the following BSG-derived preparations were used:
for inhibitory and ingredient impact effects: P1 at 1%, P3 at 2% and P4 at 4%;
for prebiotic effect: A 78 at 1%, P3 at 2% and P5 at 1%.

SkinEthic^{@} Reconstituted Human Epidermis 0.5 cm² is a reconstructed epidermis of normal human keratinocytes. Cells are grown on inert polycarbonate filters in chemically defined medium, for 17 days. The model reproduces epidermal morphology and it has been fully characterized. The good barrier function, batch reproducibility and low variability in terms of permeability for probe molecules have been described in Literature compared to human ex-vivo models. The RHE batch was tested for the absence of HIV1 and 2 antibodies, hepatitis C antibodies and hepatitis B antigen HBs. On cells from the donors, was verified the absence of bacteria, fungus and mycoplasma. The maintenance medium was tested for sterility.

### Description of Bacterial Suspension Preparation for RHE Colonization

Two days before the test, bacterial strains (S. aureus and S. epidermidis) were daily inoculated onto culture medium (BHI agar plate) and incubated at 37° C. in aerobic conditions for at least 24 h in order to obtain a fresh culture. The day of the colonization, bacterial strains have been resuspended in sterile saline solution at the required concentration range. Each starting bacterial inoculum concentration was checked by spectrophotometric OD measure and adjusted to obtain a range of about 10⁷-10⁸ UFC/mL (OD=0.1); it was then diluted 10-fold to obtain a final range concentration of 10³-10⁴ UFC/tissue for the inhibitory study and 10⁴-10⁵ UFC/tissue for prebiotic study. The starting inoculum level was also confirmed by serial decimal dilutions and viable counts on agar plates (BHI).

### Description of the Treatment and Colonization Procedure

For both inhibitory and prebiotic RHE were pre-treated twice (overnight and 1 h before colonization respectively with 50 uL and 30 uL) on the apical part (directly spread on tissue surface) and left incubated at 37° C. (with CO₂ 5%) up to the colonization. Pre-treated RHE dedicated to inhibitory and prebiotic studies were colonized with 30 μL of the bacterial suspension previously obtained (respectively with S. aureus for inhibitory study and S. epidermidis for prebiotic study) and incubated at 37° C. (with CO₂ 5%) up to the required time point (24 h). RHE sterile were not colonized pre-treated twice (overnight and 1 h before colonization respectively with 50 uL and 30 uL) on the apical part (directly spread on tissue surface) and left incubated at 37° C. (with CO₂ 5%) up to the required time point (24 h).

At the end of the time point, the apical residuals and tissues were recovered and homogenised before analyses. For both inhibitory and prebiotic studies: CFU and qRT-PCR (gene expression) for TLR-2, b-defensin2, NOD-2 were performed. It is known that:
TLR2 receptors are involved in innate immunity. Pathogen antigens recognition and activation of innate immunity via stimulation of NF-kB;
DEFB2 Induced by inflammatory stimuli and by opportunistic pathogens, supporting the hypothesis that this peptide contributes to a dynamic host defence system;
NOD-2 Plays an important role in immune system function. In some types of immune system cells, helps to protect the body against foreign invaders such as bacteria and viruses. When triggered by specific substances produced by bacteria, the NOD2 protein activates NF-kB. It also plays a role in autophagy.

See results in Table 4.

TABLE 4
Viable count for inhibitory study after 2 days using RHE model

| | S. aureus | | | |
|---|---|---|---|---|
| | Apical fraction | | Homogenate fraction | |
| | Log | Log | Log | Log |
| CODE | (Mean) | reductions | (Mean) | reductions |
| CNZ (colonized | 5.26 | // | 5.00 | // |
| control) | | | | |
| REF | <2.00 | >3.26 | <2.00 | >3.00 |
| (Chlorexidine1%) | | | | |
| P1 1% | 7.39 | −2.13 | 6.58 | −1.58 |
| P3 2% | 4.11 | 1.15 | 2.26 | 2.74 |
| P4 4% | 1.78 | 3.48 | <2.00 | >3.00 |

Colonized control (CNZ) shown a growth more than inoculum count confirming the bacterial viabilities and the test system validity.

Reference control (REF, Chlorexidine) shown no bacterial growth confirming the bacterial susceptibility to the antimicrobial substance.

The preparations P3 at 2% and P4 at 4%, compared to the colonized control have shown a capability to reduce the bacterial growth. In particular, the results indicate the following:
bold indicates the results when the number of CFUs are lower than the colonized control
bold italic indicates the results when the difference between the number of CFUs obtained in presence of the test items (P3 and P4) compared with the CFUs obtained in the colonized control is more than 0.5 Log (test item lower than control) that is considered the minimum significant reduction to define an anti-microbial effect (bacterial growth decrease).

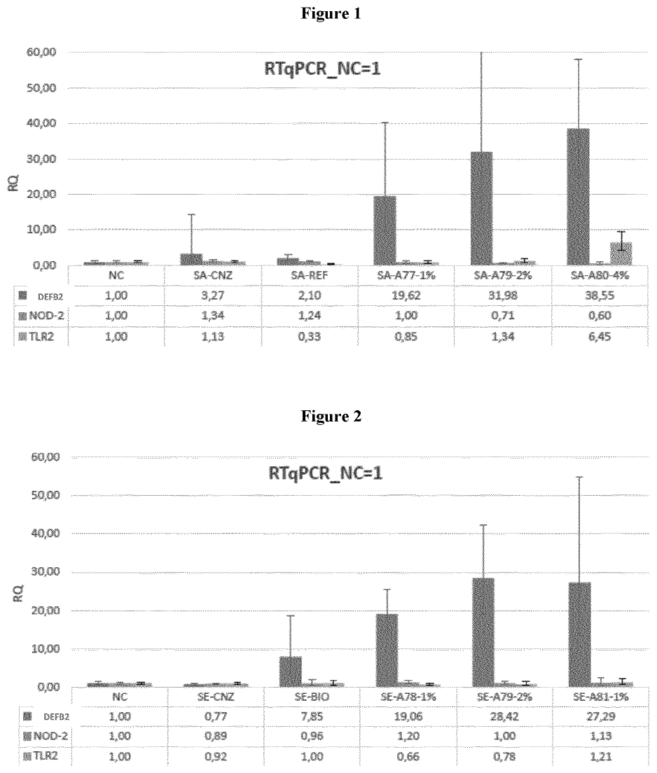

Based on the data shown in FIG. 1, DEFB2 was up-regulated in all series except for the NC (negative control): the antimicrobial protein is expressed when epidermal cells are stimulated by contact with the microorganisms and it is a dynamic component of the local innate defence system of the skin; its expression in the RHE treated with the BSG-derived ingredients suggest that these ones may have a role to induced a major innate immunity protection from infection (J.-M. Schroder, J. Harder. Molecules in focus human beta-defensin-2. Int. J. Biochem. Cell Biol., 31 (1999), pp. 645-651; P. Zanger, J. Holzer, R. Schleucher, H. Scherbaum, B. Schittek and S. Gabrysch. Severity of Staphylococcus aureus Infection of the Skin Is Associated with Inducibility of Human β-Defensin 3 but Not Human β-Defensin 2. Infection and Immunity Vol. 78, No. 7 (2010), pp 3112-3117).

NOD-2, no gene modulation was observed in all series suggesting that the biological mechanism by which the BSG-derived preparations act not based on a pro-inflammatory NfkB dependent pathway.

TRL-2 was up-regulated only in presence of the test item P4 at 4%: the protein encoded by this gene is a member of the Toll-like receptor (TLR) family which plays a fundamental role in pathogen recognition and activation of innate immunity. It is a cell-surface protein that can form heterodimers with other TLR family members to recognize molecules derived from microorganisms.

Based on the data shown in FIG. 2, DEFB2 was up-regulated in all series except for the NC (negative control) and colonized control (CNZ): the antimicrobial protein is expressed when epidermal cells are stimulated by contact with the microorganisms and it is a dynamic component of the local innate defence system of the skin; its expression in the RHE treated with the BSG-derived ingredients suggest that these ones may have a role to induced a major innate immunity protection from infection as already observed in the S. aureus case.

NOD-2, no gene modulation was observed in all series suggesting that the biological mechanism by which the BSG-derived ingredients act not based on a pro-inflammatory NfkB dependent pathway.

TLR-2 was not significantly expressed in all the samples (should be clarified during PCT)

### Conclusions

S. aureus Growth Inhibitory Study:

Based on the results obtained by the viable counts of both apical and homogenate portions after 2 days as pre-treatment, it can be stated that the best and robust results compared to colonized series (CNZ) were shown by P3 at a concentration of 2% after 2 days as pre-treatment and P4 at a concentration of 4% after 2 days as pre-treatment. The S. aureus growth inhibition was confirmed by the results on RHE colonized by the gene expression data where DEFB2 as an antimicrobial protein involved in innate immunity response was up-regulated in presence of the tested ingredients. P4 at 4% have induced a significant up-regulation of TRL-2 gene.

### Prebiotic Study:

All three BSG-derived preparations have determined the up-regulation of Befensin beta 2, gene responsible for innate immunity activation and antimicrobial properties boosting without activating pro-inflammatory pathways based on NOD-2 and TLR-2 genes.

1. A method for producing a prebiotic composition, the method comprising the steps of:
a) providing biomass;
b) optionally determining lipids, proteins and/or carbohydrates contents in the biomass;
c) optionally pre-treating the biomass, wherein the optional pre-treatment is hot water pre-treatment at about 90° C.;
d) optionally removing lipids and/or proteins from the biomass;
e) contacting the biomass with a catalyst in the presence of water and/or an organic solvent to form a reaction mixture, wherein the catalyst is an ionic polymer or a combination of ionic polymers, the ionic polymer network, a solid-supported ionic polymers and/or a polymer membrane incorporating ionic polymers;
f) heating the reaction mixture between 100° C. to 150° C. and degrading the biomass in the reaction mixture to produce a liquid phase and a solid phase, wherein the liquid phase includes the prebiotic composition, and the solid phase includes residual biomass;
g) cooling the reaction mixture to room temperature;
h) isolating at least a portion of the liquid phase from the solid phase;
i) optionally adding one or more compounds selected from bentonite, charcoal, zeolite, amorphous silica and ion exchange solvent to the isolated liquid phase; and
j) recovering the prebiotic composition from the isolated liquid phase,
wherein the ionic polymer (IP) consists of a monomer of formula I 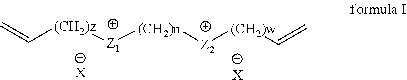
or consists of a first monomer of formula I 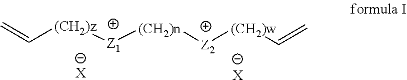
and at least one second monomer selected from the group consisting of 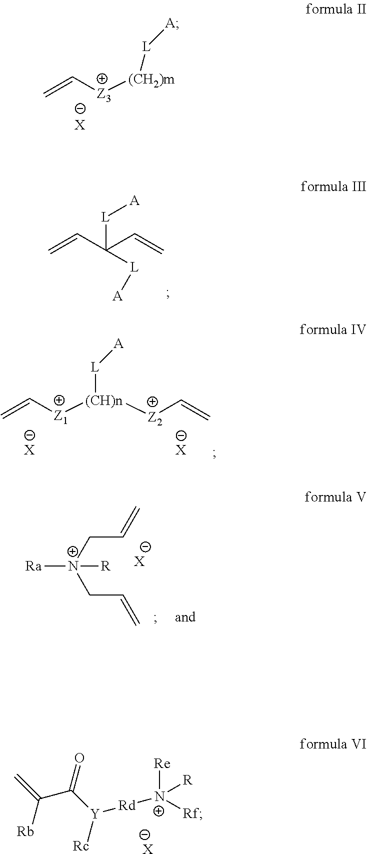
wherein:
n and m are independently selected from 1, 2, 3, 4, 5, and 6;
z and w are independently selected from zero, 1, 2, and 3;
Z₁, Z₂ and Z₃ are cations each independently selected from the group consisting of: 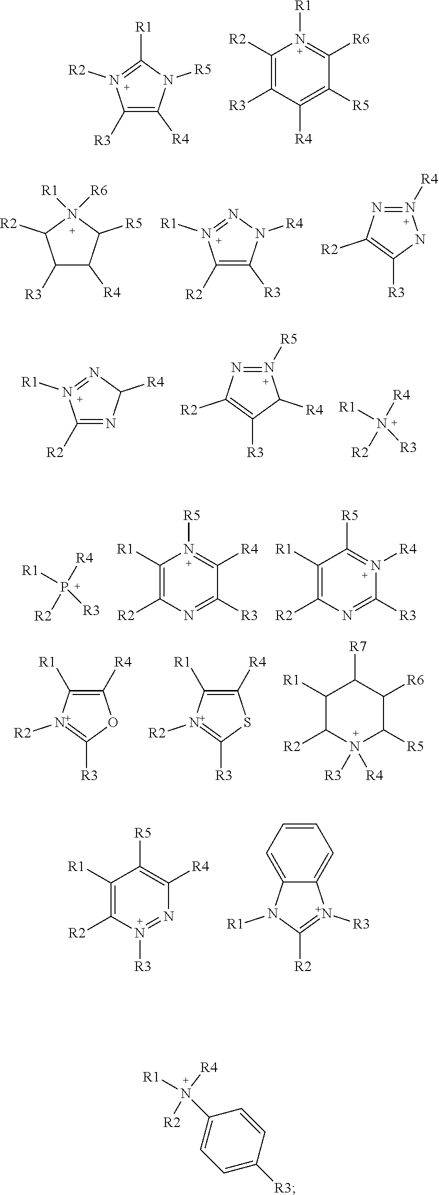
wherein:
R1, R2, R3, R4, R5, R6 and R7 are each independently selected from the group consisting of a bond, H, C₁-C₆ alkyl, C₁-C₆ allyl, —CH₂—(CH₂)p-O—(CH₂)q-CH₃, C₁-C₆ alkoxy, C₁-C₆ alkoxyalkyl, benzyl, —SO₃H, and —(CH₂)q-SO₃H, provided that two of R1, R2, R3, R4, R5, R6 and R7 are each a bond;
p and q are independently selected from zero, 1, 2, 3, 4, 5, and 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group consisting of H, —SO₃H, —COOH, —[P(═O)(OH)₂], —O—SO₃H, —O—COOH, and —O—[P(═O)(OH)₂];
A is an optional acidic group and each occurrence of A, if present, is independently selected from the group comprising-consisting of H, —SO₃H, —COOH, —[P(═O)(OH)₂], —O—SO₃H, —O—COOH, —O—[P(═O)(OH)₂], and —CH₂—COOH, provided that when z and w are zero, A is present in formula IV;
X⁻ is selected from the group comprising consisting of F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, and xylenesulfonate;
Ra is C₁-C₂₄ alkyl;
Rb and Rc are each independently H, CH₃, or absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is C₁-C₂₄ alkyl, C₅-C₁₀ aryl, or is absent;
wherein the ionic polymer network comprises cross-linked the one or more ionic polymers (IP);
wherein the solid support has at least one surface comprising the one or more ionic polymers (IP) or the ionic polymer network; and
wherein the polymer membrane incorporates the one or more ionic polymers (IP) or the ionic polymer network.

2. The method of claim 1, wherein in the ionic polymer (IP), Z₁, Z₂ and Z₃ are cations each independently selected from the group consisting of:

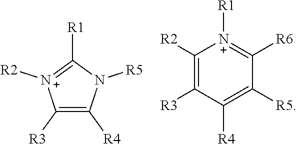

3. The method of claim 1, wherein the second monomer of formula VI is

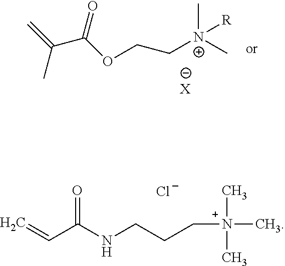

4. The method of claim 1, wherein the ionic polymer (IP) is

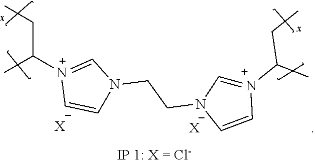

5. The method of claim 1, wherein the biomass is brewer's spent grains.

6. A prebiotic composition obtained by the method of claim 1.

7. A topical cosmetic composition comprising:
an effective amount of the prebiotic composition of claim 6, and
at least one dermatologically acceptable carrier.

8. A method for non-therapeutic treatment of the skin of a subject, the method comprising applying the topical cosmetic composition of claim 7 to the skin of the subject.

9. The method of claim 8, wherein the non-therapeutic treatment is protection and/or regeneration of the skin.

10. The method of claim 8, wherein the non-therapeutic treatment is protecting and improving the condition and/or appearance of the skin.

11. The method of claim 8, wherein the non-therapeutic treatment is maintaining skin hydration.

12. A method for modulating the growth of at least one commensal bacterium of the skin, the method comprising applying an effective amount of the topical composition of claim 7 to the skin.

13. The method of claim 12, wherein the commensal bacterium is selected from the group consisting of Propionibacterium acnes, Corynebacterium tuberculostearicum, Streptococcus mitis, Streptococcus oralis, Streptococcus pseudopneumoniae, Streptococcus sanguinis, Micrococcus luteus, Staphylococcus epidermidis, Staphylococcus capitis, and Veillonella parvula.

14. A method for preventing dysbiosis in a subject, the method comprising applying the topical composition of claim 7 to the skin of the subject.

15. A method for stimulating the immune skin system of a subject, the method comprising applying the topical composition of claim 7 to the skin of the subject.
